(19) United States
(12) Patent Application Publication
(10) Pub. No.: US 2025/0359826 A1
(43) Pub. Date: Nov. 27, 2025

(54) REGIONAL OXIMETRY SIGNAL PROCESSOR

(71) Applicant: MASIMO CORPORATION, Irvine, CA (US)

(72) Inventors: Ammar Al-Ali, San Juan Capistrano, CA (US); David Dalke, Rancho Santa Margarita, CA (US); Eric Karl Kinast, Santa Ana, CA (US); Walter M. Weber, Laguna Hills, CA (US); Yuying Song, Irvine, CA (US); Faisal Kashif, Foothill Ranch, CA (US); Atiyeh Ghoreyshi, Foster City, CA (US); Kevin Forrest, Rancho Santa Margarita, CA (US)

(21) Appl. No.: 19/232,633

(22) Filed: Jun. 9, 2025

Related U.S. Application Data
(63) Continuation of application No. 17/448,340, filed on Sep. 21, 2021, now Pat. No. 12,357,237.
Application No. 17/448,340 is a continuation of application No. 16/121,388, filed on Sep. 4, 2018, now Pat. No. 11,147,518.
Application No. 16/121,388 is a continuation of application No. 14/507,715, filed on Oct. 6, 2014, now abandoned.
(60) Provisional application No. 61/887,881, filed on Oct. 7, 2013.
(60) Provisional application No. 61/887,878, filed on Oct. 7, 2013.
(60) Provisional application No. 61/887,856, filed on Oct. 7, 2013.
(60) Provisional application No. 61/887,883, filed on Oct. 7, 2013.
(60) Provisional application No. 62/012,170, filed on Jun. 13, 2014.

(51) Int. Cl. A61B 5/00; A61B 5/1455

(52) U.S. Cl. CPC A61B 5/7228; A61B 5/14552; A61B 5/14553; A61B 5/7225; A61B 5/7278; A61B 5/742

(57) ABSTRACT

A regional oximetry system comprises a pod having a pod housing defining a sensor end and an opposite monitor end. A dual sensor connector is in electrical communication with the sensor end of the pod housing. A monitor connector is in electrical communication with the monitor end of the pod housing. An analog board is disposed within the pod housing and is in electrical communications with the dual sensor connector. The analog board receives and digitizes sensor signals from at least one optical sensor plugged into the dual sensor connector. A digital board is disposed within the pod housing and in electrical communications with the analog board and the monitor connector. A digital signal processor (DSP) is mounted on the digital board and implements a regional oximetry signal processor so as to receive digitized sensor signals from the analog board, derive regional oximetry parameters from the digitized sensor signals and communicate the regional oximetry parameters to the monitor connector for display on an attached monitor.

### PRIORITY CLAIM TO RELATED APPLICATIONS

Any and all applications for which a foreign or domestic priority claim is identified in the Application Data Sheet as filed with the present application are hereby incorporated by reference under 37 CFR 1.57. The present application is a continuation of U.S. application Ser. No. 17/448,340, filed Sep. 21, 2021, titled Regional Oximetry Signal Processor, which is a continuation of U.S. application Ser. No. 16/121,388 filed Sep. 4, 2018, titled Regional Oximetry Signal Processor, which is a continuation of U.S. application Ser. No. 14/507,715, filed Oct. 6, 2014, titled Regional Oximetry Signal Processor, which claims priority benefit under 35 U.S.C. § 119(e) to U.S. Provisional Patent Application Ser. No. 62/012,170, filed Jun. 13, 2014, titled Peel-Off Resistant Regional Oximetry Sensor, U.S. Provisional Patent Application Ser. No. 61/887,881 filed Oct. 7, 2013, titled Regional Oximetry Signal Processor; U.S. Provisional Patent Application Ser. No. 61/887,878 filed Oct. 7, 2013, titled Regional Oximetry Pod; U.S. Provisional Patent Application Ser. No. 61/887,856 filed Oct. 7, 2013, titled Regional Oximetry Sensor; and U.S. Provisional Patent Application Ser. No. 61/887,883 filed Oct. 7, 2013, titled Regional Oximetry User Interface; all of the above-referenced provisional patent applications are hereby incorporated in their entireties by reference herein.

### BACKGROUND

Pulse oximetry is a widely accepted noninvasive procedure for measuring the oxygen saturation level of arterial blood, an indicator of a person's oxygen supply. A typical pulse oximetry system utilizes an optical sensor attached to a fingertip to measure the relative volume of oxygenated hemoglobin in pulsatile arterial blood flowing within the fingertip. Oxygen saturation (SpO2), pulse rate and a plethysmograph waveform, which is a visualization of pulsatile blood flow over time, are displayed on a monitor accordingly.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entirety by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (Cercacor) and all incorporated in their entirety by reference herein. Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY

Regional oximetry, also referred to as tissue oximetry and cerebral oximetry, enables the continuous assessment of tissue oxygenation beneath the sensor. Regional oximetry helps clinicians detect regional hypoxemia that pulse oximetry alone can miss. In addition, the pulse oximetry capability in regional oximetry sensors can automate a differential analysis of regional to central oxygen saturation. Regional oximetry monitoring is as simple as applying regional oximetry sensors to any of various body sites including the forehead, forearms, chest, upper thigh, upper calf or calf, to name a few, and connecting one or two pods each connected to one or two sensors to a monitor through, for example, USB ports.

One aspect of a regional oximetry system comprises a pod having a pod housing defining a sensor end and an opposite monitor end. A dual sensor connector is in electrical communication with the sensor end of the pod housing. A monitor connector is in electrical communication with the monitor end of the pod housing. An analog board is disposed within the pod housing and in electrical communications with the dual sensor connector so as to receive sensor signals from at least one optical sensor plugged into the dual sensor connector and so as to digitize the sensor signals. A digital board is disposed within the pod housing and in electrical communications with the analog board and the monitor connector. A digital signal processor (DSP) is mounted on the digital board so as to receive the digitized sensor signals from the analog board, derive regional oximetry parameters from the digitized sensor signals and communicate the regional oximetry parameters to the monitor connector for display on an attached monitor.

In various embodiments, a regional oximetry signal processor executes on the DSP. The regional oximetry signal processor has a front-end and a back-end. The front-end inputs the digitized sensor signals and outputs demodulated/decimated sensor data responsive to the sensor signals. The back-end receives the demodulated/decimated sensor data and outputs regional oximetry parameters responsive to the sensor data. A USB interface receives the regional oximetry parameters from the back-end and outputs the regional oximetry parameters to a USB cable. The USB cable is in communications with a USB connector configured to plug into a USB port of a patient monitor. The analog board has at least one switch matrix and the switch matrix is in communications with the dual sensor connector so as drive sensor emitters of at least one optical sensor plugged into the dual sensor connector. The analog board has analog-to-digital converters (ADCs) in communications with at least one detector of the optical sensor plugged into the dual sensor connector so as to generate the digitized sensor signals. A DSP bus is in communications with the DSP and the ADCs are in communications with the DSP bus so as to communicate the digitized sensor signals to the DSP. A shift register on the digital board is in communications with the switch matrix so as to individually select the sensor emitters to drive.

Another aspect of regional oximetry system is a signal processing method comprising attaching a first regional oximetry sensor and a second regional oximetry sensor to a patient. The sensors being in close proximity, the first sensor having a plurality of first emitters and the second sensor having a plurality of second emitters. The first sensor and the second sensor are operated concurrently so as to derive regional oximetry parameters that are comparable in time. The first sensor and the second sensor are modulated so that the on periods of the first emitters do not overlap with the on periods of the second emitters.

In various embodiments, the first emitters and the second emitters are modulated so that the on periods of the first emitters have the same duty cycle as the on periods of the second emitters. The modulation of the first emitters and the second emitters are synchronized to the same n-bin period, where n is an integer and a bin is defined as a fixed time interval. A first on-period and a first off-period are defined for the first emitters. A second on-period and a second off-period are defined for the second emitters. The first on-period is not equal to the second on-period, and the first off-period is not equal to the second off-period.

In further embodiments, the n-bin period is 73 bins. The first emitters are on for 3 cycles of 73 bins totaling 219 bins. The second emitters are on for 5 cycles of 73 bins totaling 365 bins. The first emitters have a 15 bin on-time and a 219 bin off-time for a 6.85% duty cycle, and the second emitters have a 25 bin on-time and a 365 bin off-time for a 6.85% duty cycle.

A further aspect of a regional oximetry signal processing system is a plurality of regional oximetry sensor means for optically probing both near-field and far-field portions of a tissue site. An analog means is for electrically driving the sensor means and receiving sensor data from the sensor means. A digital means is for deriving regional oxygen saturation parameters from the sensor data. A pod means is for housing the analog means and the digital means. A plurality of sensor cable means is for electrically interconnecting the plurality of sensor means to the pod means. A pod cable means is for interconnecting the pod means to a monitoring device.

In various embodiments, the pod means further comprises a signal processing means for converting the sensor data to physiological parameters. The signal processing means comprises a front-end means for demodulating the sensor signals and decimating the demodulated sensor signals in time. The signal processing means further comprises a back-end means for deriving a regional oxygen saturation from the decimated and demodulated sensor signals. The analog means comprises a plurality of switch matrix means for driving the sensor means and a plurality of analog-to-digital converter means for receiving the sensor data.

### BRIEF DESCRIPTION OF THE DRAWINGS

The drawings and following associated descriptions are provided to illustrate embodiments of the present disclosure and do not limit the scope of the claims. Corresponding numerals indicate corresponding parts, and the leading digit of each numbered item indicates the first figure in which an item is found.

FIG. 1 is a general block diagram of a regional oximetry signal processing system including regional oximetry sensors, sensor pods, signal processors and a patient monitor;

FIGS. 2A-2B are perspective views of various regional oximetry sensor and sensor pod embodiments;

FIG. 3 is a cross-sectional view of a regional oximetry sensor attached to a tissue site illustrating corresponding near-field and far-field emitter-to-detector optical paths;

FIG. 4 is a general block diagram of a regional oximetry pod housing a regional oximetry signal processor;

FIG. 5 is a general block diagram of a regional oximetry signal processor;

FIG. 6 is a detailed block diagram of a front-end signal processor;

FIG. 7 is a detailed block diagram of a back-end signal processor;

FIG. 8 is a detailed block diagram of the emitter drive for dual regional sensors;

FIG. 9 is a detailed block diagram of the detector interface for dual regional sensors;

FIG. 10 is a dual-sensor anti-crosstalk LED timing diagram;

FIG. 11 is a regional oximetry monitor display that provides user I/O for indicating the placement of up to four sensors on a patient; and

FIG. 12 is a regional oximetry parameter display accommodating inputs from four regional oximetry sensors.

### DETAILED DESCRIPTION

Aspects of the disclosure will now be set forth in detail with respect to the figures and various embodiments. One of skill in the art will appreciate, however, that other embodiments and configurations of the devices and methods disclosed herein will still fall within the scope of this disclosure even if not described in the same detail as some other embodiments. Aspects of various embodiments discussed do not limit the scope of the disclosure herein, which is instead defined by the claims following this description.

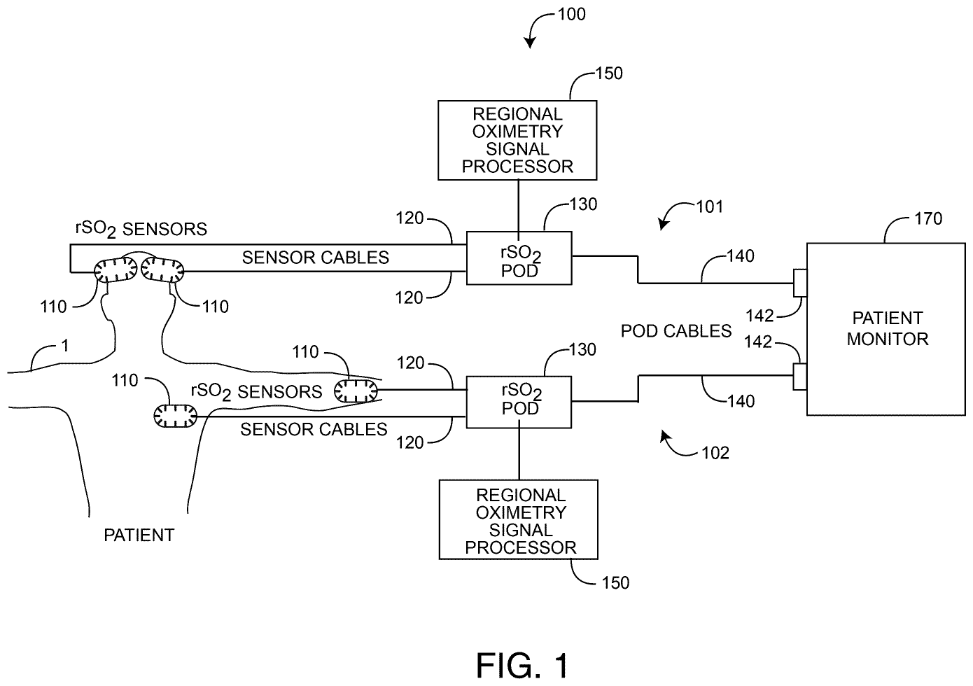

FIG. 1 generally illustrates a regional oximetry signal processing system 100 including pod assemblies 101, 102 each communicating with an array of regional oximetry sensors 110 via sensor cables 120. The sensors 110 are attached to various patient 1 locations, with one or two regional oximetry pods 130 and a corresponding number of pod cables 140 providing communications between the pods 130 and a patient monitor 170. Advantageously, regional oximetry signal processors 150 housed in each of the pods 130 perform the algorithmic processing normally associated with patient monitors and/or corresponding monitor plug-ins so as to derive various regional oximetry (rSO₂) parameters. Further, in an embodiment, each signal processor 150 utilizes USB communication protocols and connectors 142 to easily integrate with a third party monitor 170. Monitor 170 may range from a relatively “dumb” display device to a relatively “intelligent” multi-parameter patient monitor so as to display physiological parameters indicative of health and wellness.

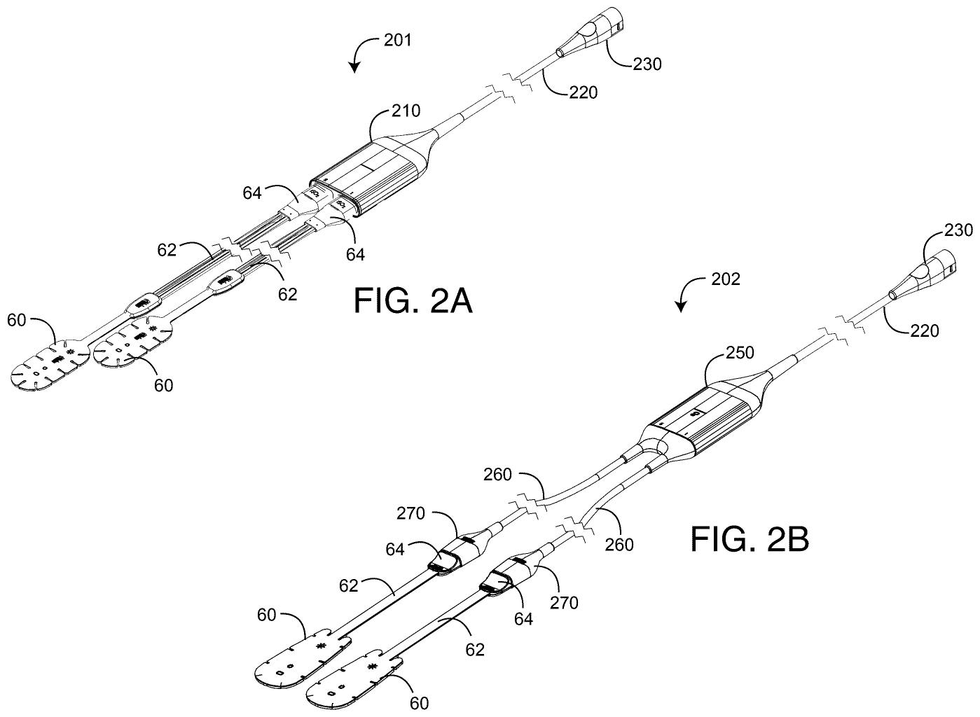

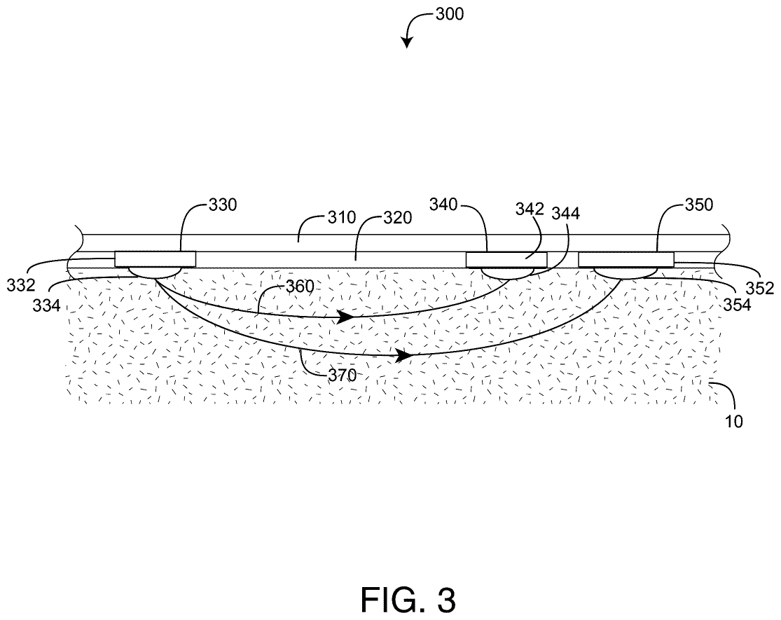

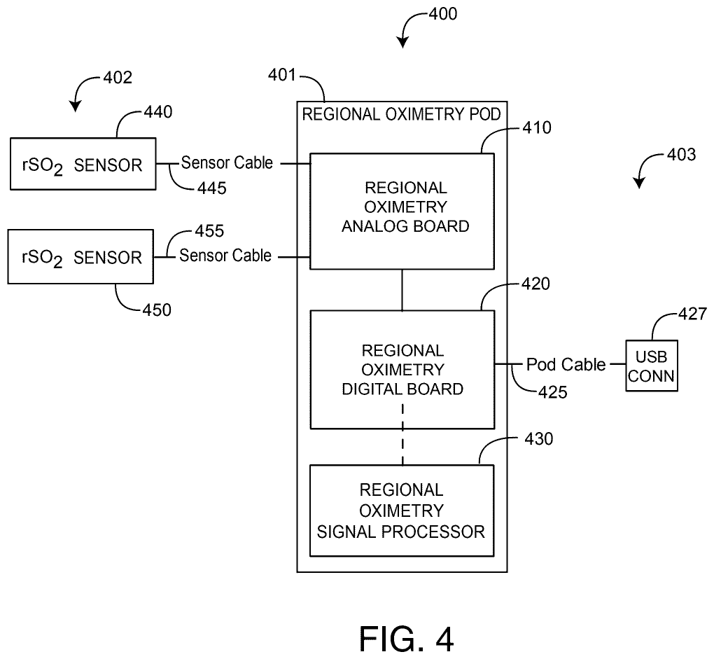

FIGS. 2A-2B illustrate sensor pods 201, 202. As shown in FIG. 2A, a sensor pod 201 embodiment has a pod housing 210 with pod sockets (not visible) recessed into the pod housing 210. RSO₂ sensors 60 have sensor cables 62 extending between the sensor 60 and sensor plugs 64. The sensor plugs 64 insert into the pod sockets so as communicate sensor signals between the sensor 60 and pod analog and digital boards (not visible) within the pod housing 210. Pod boards are described in detail with respect to FIGS. 4-9, below. Sensor optics and corresponding sensor signals are described with respect to FIG. 3, below.

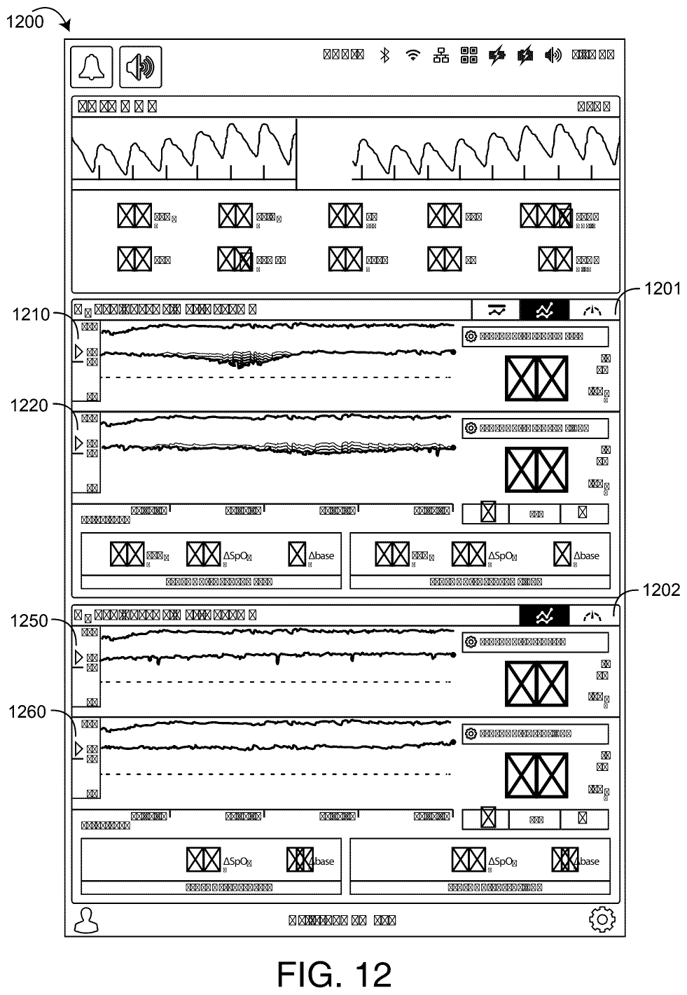

FIG. 3 illustrates a regional oximetry sensor 300 attached to a tissue site 10 so as to generate near-field 360 and far-field 370 emitter-to-detector optical paths through the tissue site 10. The resulting detector signals are processed so as to calculate and display oxygen saturation (SpO₂), delta oxygen saturation (ASpO₂) and regional oxygen saturation (rSO₂), as shown in FIG. 12, below. The regional oximetry sensor 300 has a flex circuit layer 310, a tape layer 320, an emitter 330, a near-field detector 340 and a far-field detector 350. The emitter 330 and detectors 340, 350 are mechanically and electrically connected to the flex circuit 310. The tape layer 320 is disposed over and adheres to the flex circuit 310. Further, the tape layer 320 attaches the sensor 300 to the skin 10 surface.

As shown in FIG. 3, the emitter 330 has a substrate 332 mechanically and electrically connected to the flex circuit 310 and a lens 334 that extends from the tape layer 320. Similarly, each detector 340, 350 has a substrate 342, 352 and each has a lens 344, 354 that extends from the tape layer. In this manner, the lenses 334, 344, 354 press against the skin 10, advantageously maximizing the optical transmission and reception of the emitter 330 and detectors 340, 350.

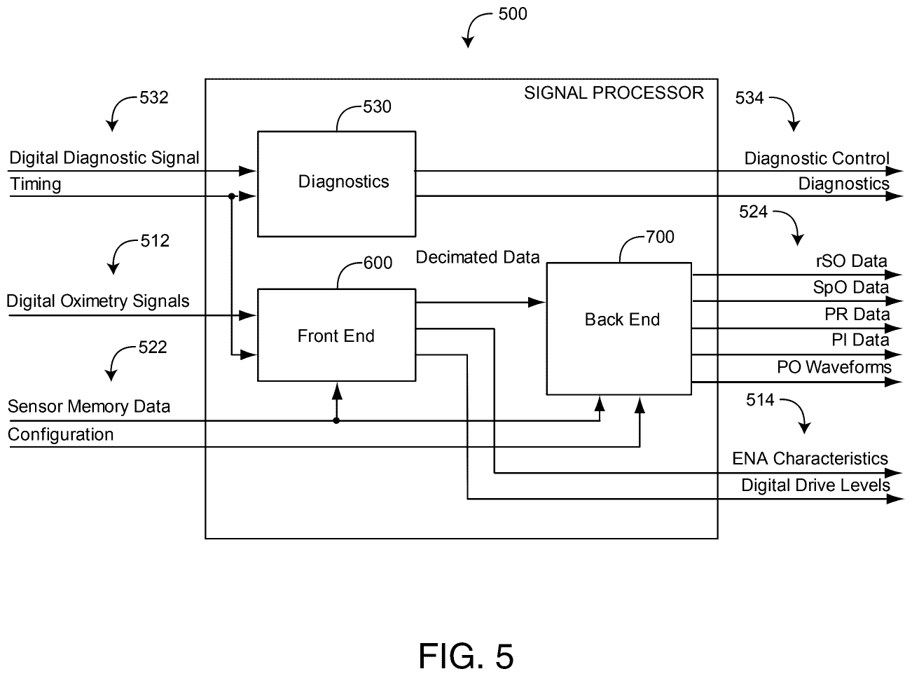

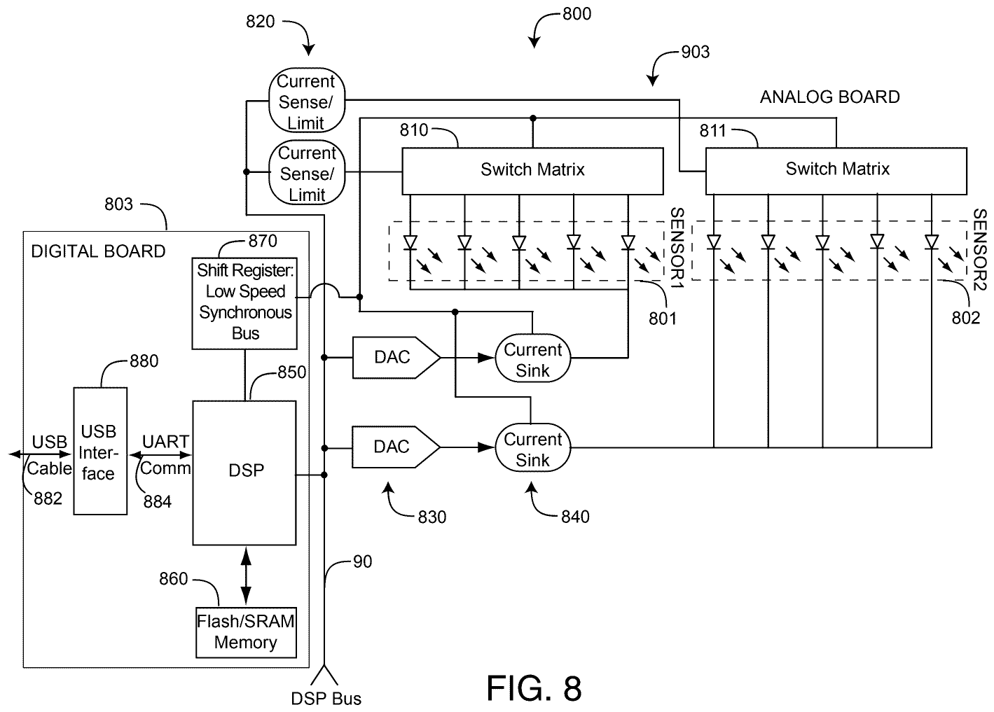

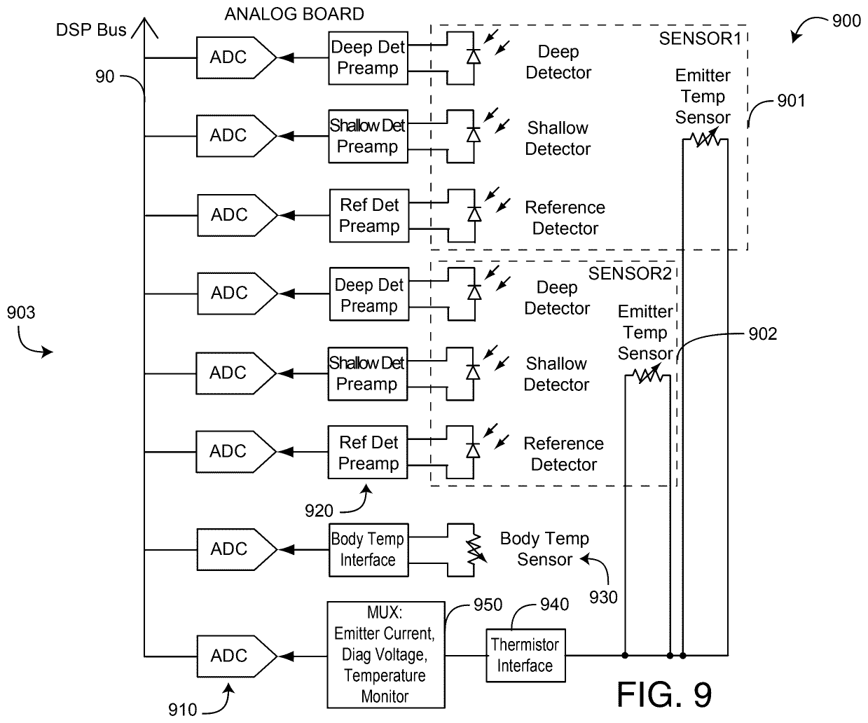

FIG. 4 illustrates a regional oximetry pod 401 that houses a regional oximetry analog board 410 and a regional oximetry digital board 420. A regional oximetry signal processor 430 executes on a digital signal processor (DSP) residing on the digital board 420. The regional oximetry signal processor 430 is described in detail with respect to FIGS. 5-7, below. The regional oximetry analog board 410 and digital board 420 are described in detail with respect to FIGS. 8-9, below.

As shown in FIG. 4, on the patient side 402, the regional oximetry analog board 410 communicates with one or more regional oximetry (rSO₂) sensors 440, 450 via one or more sensor cables 445, 455. On the caregiver side 403, a pod cable 425 has a USB connector 427 so as to provide a standard interface between the digital board 420 and a monitor 170 (FIG. 1).

Also shown in FIG. 4, the analog board 410 and the digital board 420 enable the pod 401 itself to perform the sensor communications and signal processing functions of a conventional patient monitor. This advantageously allows pod-derived regional oximetry parameters to be displayed on a variety of monitors ranging from simple display devices to complex multiple parameter patient monitoring systems via the simple USB interface 427.

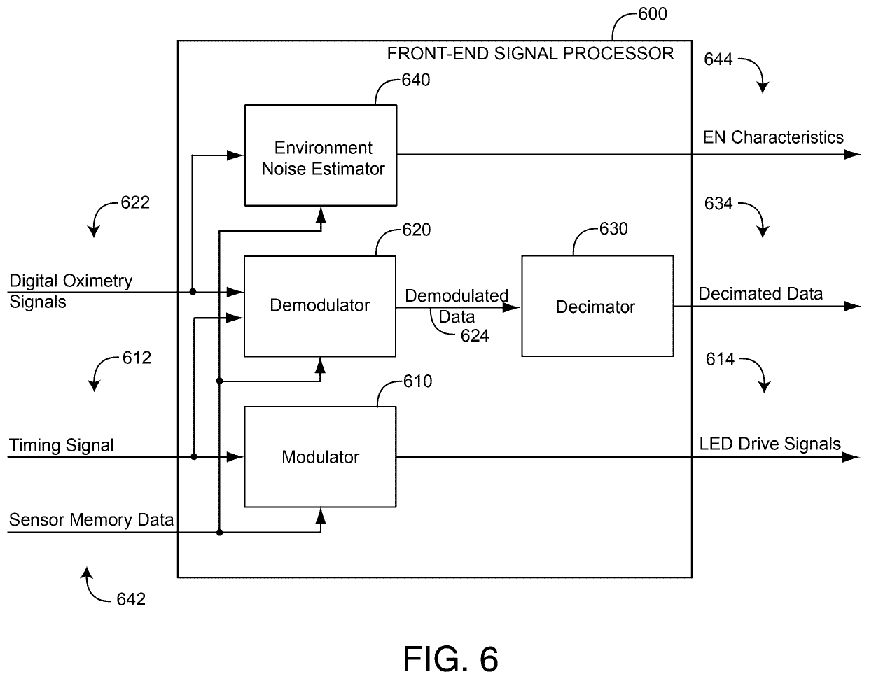

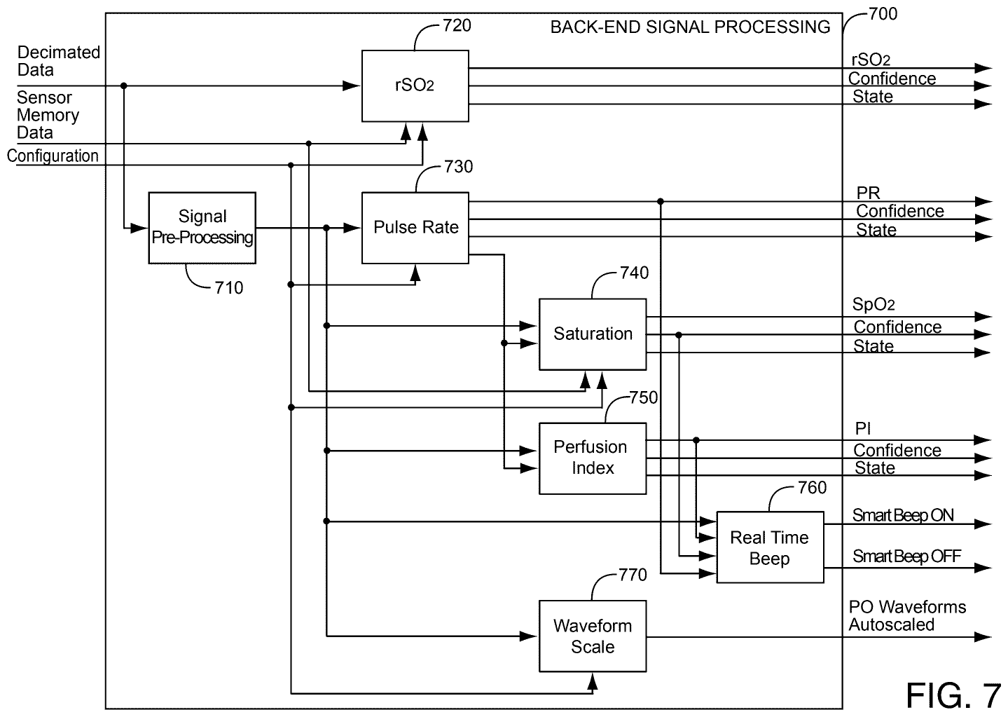

FIG. 5 generally illustrates a regional oximetry signal processor 500 having a front-end signal processor 600, a back-end signal processor 700 and diagnostics 530. The front end 600 controls LED modulation, detector demodulation and data decimation, as described in detail with respect to FIG. 6, below. The back-end 700 computes sensor parameters from the decimated data, as described in detail with respect to FIG. 7, below. The diagnostics 530 analyze data corresponding to various diagnostic voltages within or external to the digital board so as to verify system integrity.

FIG. 6 illustrates a front-end signal processor 600 having a modulator 610, a demodulator 620, a decimator 630 and an environmental noise estimator 640. The modulator 610 multiplexes drive signals to various wavelength LEDs 614. The demodulator 620 de-multiplexes the signals detected after tissue site attenuation to generate demodulated data 624. The decimator 630 reduces the sample rate of the resulting demodulated data 624 to that of the decimated data 634. The environmental noise estimator 640 characterizes the amount of environmental noise (EN) 644 in the detected signals, such as from ambient light and electromagnetic interferences like electro surgery instruments.

FIG. 7 illustrates regional oximetry back-end signal processor 700 that computes regional oximetry physiological parameters. The signal pre-processor 710 compensates for variations in LED intensities. rSO₂ 720 calculates regional oxygen saturation, its confidence and its state (an indicator of the calculation method). Pulse rate 730 calculates pulse rate, confidence and its state. Saturation 740 calculates arterial oxygen saturation, confidence and state. Perfusion index 750 calculates perfusion, confidence and its state. Real time beep 760 generates an audible tone (beep) according to a detected arterial pulse. Waveform scale 770 scales the input pleth using a linear map that maps the range of the input signal to the range of the output signal.

FIGS. 8-9 illustrate a regional oximetry signal processor embodiment 800, 900 having a digital board 803 (FIG. 8) and an analog board 903 (FIGS. 8-9) in communications with up to two regional oximetry sensors 801, 802 (FIG. 8); 901, 902 (FIG. 9). The digital board 803 (FIG. 8) has a DSP 850 in communications with an external monitor via a USB cable 882 and corresponding UART communications 884. The DSP 850 is also in communications with the sensors 801-802, 901-902 via DACs 830 and ADCs 910 on the analog board 903.

As shown in FIGS. 8-9, sensor emitters 801, 802 are driven from the analog board 903 under the control of the digital board DSP 850 via a shift register 870. Each regional sensor 801-802, 901-902 has a shallow detector and a deep detector. Further, each sensor 801-802, 901-902 may have a reference detector and an emitter temperature sensor.

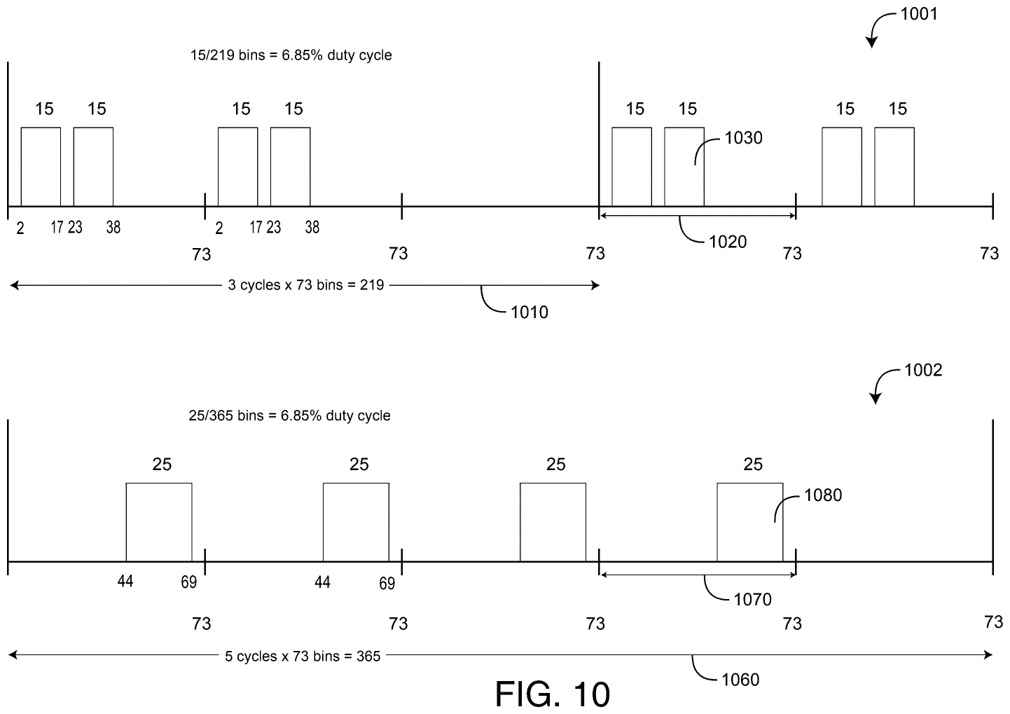

FIG. 10 illustrates dual sensor modulation timing 1001, 1002 so as to avoid sensor crosstalk. A regional oximetry sensor 60 (FIGS. 2A-B) has a deep detector 350 (FIG. 3) and a shallow detector 340 (FIG. 3). Multiple sensors 60 (FIGS. 2A-B) may be placed in close proximity on a patient, such as a left forehead placement for a first sensor and a right forehead placement for a second sensor. As such, light from the first sensor's emitters may be received by the second sensor's detectors if both sensor's emitters are cycled on at the same time. A modulation schema 1001, 1002 provides for two sensor's emitters to be advantageously non-overlapping in time while maintaining the same duty cycle per emitter.

As shown in FIG. 10, each of two proximate sensor's emitters 1001, 1002 are cycled on and off in sync to a fixed period 1010, 1060 measured in bins. In an embodiment, each sensor 1001, 1002 has four emitters and each is synchronized to a 73-bin period 1020, 1070. A first sensor 1001 utilizes an emitter on period of 15 bins 1030 over a 3×73-bin cycle, with the last cycle off. A second sensor 1002 utilizes an emitter on period of 25 bins 1080 over a 5×73-bin cycle, again with the last cycle off. As shown, the on-periods for each emitter 1030, 1080 are always non-overlapping, so as to avoid crosstalk between adjacent sensors. Further, each sensor advantageously has the same overall duty cycle of 6.85%.

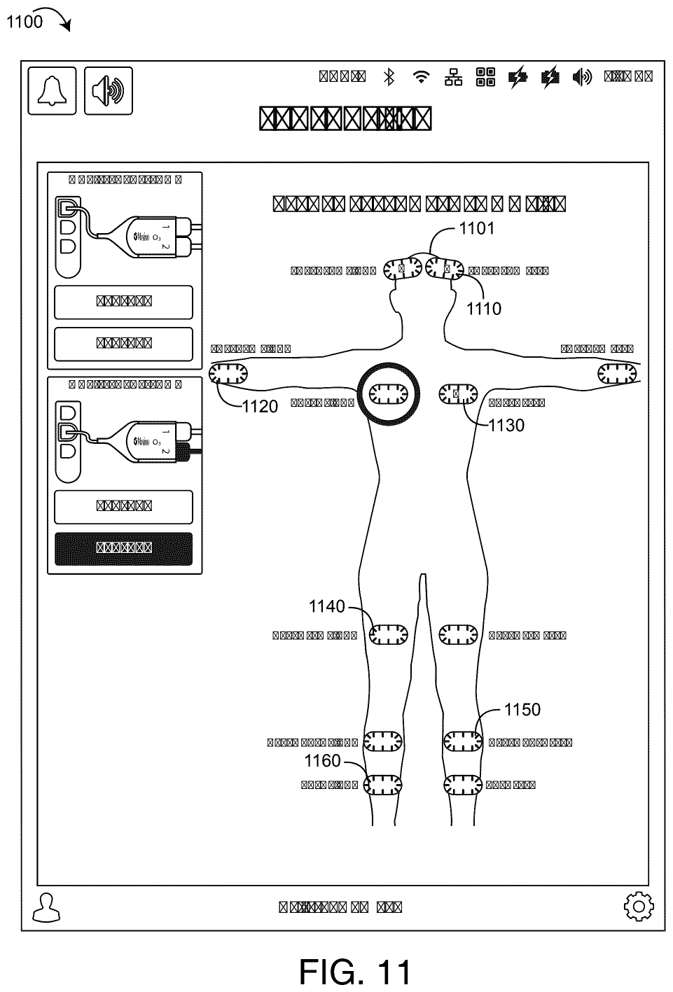

FIG. 11 illustrates a user I/O display 1100 for indicating the placement of up to four sensors on a patient. An adult form 1101 is generated on the display. Between one and four sensor sites can be designated on the adult form 1101, including left and right forehead 1110, forearm 1120, chest 1130, upper leg 1140, upper calf 1150 and right calf 1160 sites. Accordingly, between one and four sensors 110 (FIG. 1) can be located on these sites. A monitor in communication with these sensors then displays between one and four corresponding regional oximetry graphs and readouts, as described with respect to FIG. 12, below.

FIG. 12 illustrates a regional oximetry parameter display 1200 embodiment for accommodating up to four regional oximetry sensor inputs. In this particular example, a first two sensor display 1201 is enabled for monitoring a forehead left site 1210 and a forehead right site 1220. A second two sensor display 1202 is enabled for monitoring a chest left site 1250 and a chest right site 1260.

A regional oximetry signal processor has been disclosed in detail in connection with various embodiments. These embodiments are disclosed by way of examples only and are not to limit the scope of the claims herein. One of ordinary skill in art will appreciate many variations and modifications.

Embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. In addition, the foregoing embodiments have been described at a level of detail to allow one of ordinary skill in the art to make and use the devices, systems, etc. described herein. A wide variety of variation is possible. Components, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, engines, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

1.-20. (canceled)

21. A regional oximetry system comprising:
a regional oximetry sensor comprising:
a sensor body configured to be secured to a forehead of a subject;
at least one emitter operably positioned by the sensor body and configured to emit light towards skin of the subject's forehead;
at least one detector operably positioned by the sensor body and configured to detect light reflected from the subject's skin and generate one or more signals based on the detected light; and
a sensor cable connected to the sensor body;
a cable adapter for receiving and processing said one or more signals generated by the at least one detector of the regional oximetry sensor, the cable adapter comprising:
a housing comprising an interior, a first end, and a second end opposite the first end;
a first connector arranged at the first end of the housing, the first connector configured to removably connect to the sensor cable of the regional oximetry sensor;
an adapter cable integrally formed with and extending outward from the second end of the housing;
a second connector formed at an end of the adapter cable;
an analog board arranged within the interior of the housing and configured to receive said one or more signals from the regional oximetry sensor when the regional oximetry sensor is connected to the first connector of the cable adapter, the analog board further configured to digitize said one or more signals;
a digital board arranged within the interior of the housing and in electrical communication with the analog board; and
a digital signal processor in electrical communication with the digital board, the digital signal processor configured to receive the digitized one or more signals and determine one or more physiological parameters of the subject based on said digitized one or more signals; and
a monitoring device configured to removably connect to the second connector of the cable adapter and receive the one or more physiological parameters from the cable adapter, the monitoring device further configured to display information relating to said one or more physiological parameters.

22. The regional oximetry system of claim 21, wherein the regional oximetry sensor comprises a plurality of emitters, and wherein the digital signal processor is further configured to control individual selection of the plurality of emitters driven from the analog board when the regional oximetry sensor is connected to the first connector of the cable adapter.

23. The regional oximetry system of claim 21, wherein the digital signal processor is configured to:
generate demodulated/decimated sensor data responsive to the digitized one or more signals; and
determine, based on the demodulated/decimated sensor data, the one or more regional oximetry parameters of the subject.

24. The regional oximetry system of claim 21, the analog board comprising a switch matrix, the switch matrix in communication with the first connector, the switch matrix configured to drive the at least one emitter of the regional oximetry sensor when the regional oximetry sensor is connected to the first connector.

25. The regional oximetry system of claim 24, wherein the at least one emitter comprising a plurality of emitters, and wherein the digital board comprises a shift register in communication with the switch matrix, the shift register and the switch matrix configured to individually select one of the plurality of emitters.

26. The regional oximetry system of claim 21, wherein the cable adapter comprises a bus in communication with the digital signal processor and analog board.

27. The regional oximetry system of claim 21, wherein the cable adapter includes no user inputs or display.

28. A regional oximetry system comprising:
a regional oximetry sensor comprising:
a sensor body configured to be secured to a forehead of a subject;
at least one emitter operably positioned by the sensor body and configured to emit light towards skin of the subject's forehead;
at least one detector operably positioned by the sensor body and configured to detect light reflected from the subject's skin and generate one or more signals based on the detected light; and
a sensor cable connected to the sensor body;
a cable adapter comprising:
a housing comprising an interior, a first end, and a second end opposite the first end;
a connector arranged at the first end of the housing, the connector configured to removably connect to the sensor cable of the regional oximetry sensor;
an adapter cable integrally formed with and extending outward from the second end of the housing, the adapter cable configured to removably connect to a monitoring device;
an analog board arranged within the interior of the housing and configured to receive said one or more signals from the regional oximetry sensor when the regional oximetry sensor is connected to the first connector of the cable adapter, the analog board further configured to digitize said one or more signals;
a digital board arranged within the interior of the housing and in electrical communication with the analog board; and
a digital signal processor in electrical communication with the digital board, the digital signal processor configured to receive the digitized one or more signals and determine one or more physiological parameters of the subject based on said digitized one or more signals, wherein the digital signal processor is further configured to transmit said one or more physiological parameters to said monitoring device when the adapter cable is connected to the monitoring device.

29. The regional oximetry system of claim 28, wherein the regional oximetry sensor comprises a plurality of emitters, and wherein the digital signal processor is further configured to control individual selection of the plurality of emitters driven from the analog board when the regional oximetry sensor is connected to the first connector of the cable adapter.

30. The regional oximetry system of claim 28, wherein the digital signal processor is configured to:
generate demodulated/decimated sensor data responsive to the digitized one or more signals; and
determine, based on the demodulated/decimated sensor data, the one or more regional oximetry parameters of the subject.

31. The regional oximetry system of claim 28, the analog board comprising a switch matrix, the switch matrix in communication with the first connector, the switch matrix configured to drive the at least one emitter of the regional oximetry sensor when the regional oximetry sensor is connected to the first connector.

32. The regional oximetry system of claim 31, wherein the at least one emitter comprising a plurality of emitters, and wherein the digital board comprises a shift register in communication with the switch matrix, the shift register and the switch matrix configured to individually select one of the plurality of emitters.

33. The regional oximetry system of claim 28, wherein the cable adapter comprises a bus in communication with the digital signal processor and analog board.

34. The regional oximetry system of claim 28, wherein the cable adapter includes no user inputs or display.

35. A regional oximetry system comprising:
a cable adapter comprising:
a housing comprising an interior, a first end, and a second end opposite the first end;
a connector arranged at the first end of the housing, the connector configured to removably connect to a regional oximetry sensor;
an adapter cable integrally formed with and extending outward from the second end of the housing, the adapter cable configured to removably connect to a monitoring device;
an analog board arranged within the interior of the housing and configured to receive one or more signals from the regional oximetry sensor when the regional oximetry sensor is connected to the connector of the cable adapter, the analog board further configured to digitize said one or more signals;
a digital board arranged within the interior of the housing and in electrical communication with the analog board; and
a digital signal processor in electrical communication with the digital board, the digital signal processor configured to receive the digitized one or more signals and determine one or more physiological parameters of a subject based on said digitized one or more signals, wherein the digital signal processor is further configured to transmit said one or more physiological parameters to said monitoring device when the adapter cable is connected to the monitoring device.

36. The regional oximetry system of claim 35, wherein the regional oximetry sensor comprises a plurality of emitters, and wherein the digital signal processor is further configured to control individual selection of the plurality of emitters driven from the analog board when the regional oximetry sensor is connected to the first connector of the cable adapter.

37. The regional oximetry system of claim 35, wherein the digital signal processor is configured to:
generate demodulated/decimated sensor data responsive to the digitized one or more signals; and
determine, based on the demodulated/decimated sensor data, the one or more regional oximetry parameters of the subject.
